(19) United States
(12) Patent Application Publication
(10) Pub. No.: US 2020/0388357 A1
(43) Pub. Date: Dec. 10, 2020

(54) SHARED REVOCATION LEDGER FOR DATA ACCESS CONTROL

(71) Applicant: 3M INNOVATIVE PROPERTIES COMPANY, St. Paul, MN (US)

(72) Inventors: William Rey Johnson, Salt Lake City, UT (US); Senthil Nachimithu, Salt Lake City, UT (US)

(21) Appl. No.: 15/929,894

(22) Filed: May 28, 2020

Related U.S. Application Data
(60) Provisional application No. 62/857,693, filed on Jun. 5, 2019.

(51) Int. Cl. G16H 10/60; H04L 9/06; H04L 9/32

(52) U.S. Cl. CPC G16H 10/60; H04L 9/3263; H04L 9/0637

(57) ABSTRACT

A computer implemented method includes receiving a request at a provider for protected data, the request including a unique identifier corresponding to an authorization to access the protected data or a participant identifier, searching a revocation ledger, denying the request for protected data in response to the unique identifier being stored in the revocation ledger wherein the revocation ledger comprises cryptographically linked blocks of revocations of previous documents granting access to the protected data, denying the request for protected data in response to the participant identifier being stored in the revocation ledger, and granting the request for the protected data in response to neither the unique identifier nor that participant identifier being stored in the revocation ledger.

### BACKGROUND

Currently, patients grant (authorize) or revoke access to their data for each organization or system individually, i.e., there is no central authority managing the authorization records. Because there is no central authority managing the authorization records (i.e., access granted), and a user may not know exactly where the previously issued authorization has been stored, it becomes challenging to track what access has been authorized or revoked.

### SUMMARY

A computer implemented method includes receiving a request at a provider for protected data, the request including a unique identifier corresponding to an authorization to access the protected data or a participant identifier, searching a revocation ledger, denying the request for protected data in response to the unique identifier being stored in the revocation ledger wherein the revocation ledger comprises cryptographically linked blocks of revocations of previous documents granting access to the protected data, denying the request for protected data in response to the participant identifier being stored in the revocation ledger, and granting the request for the protected data in response to neither the unique identifier nor that participant identifier being stored in the revocation ledger.

### BRIEF DESCRIPTION OF THE DRAWINGS

FIG. 1 is a block diagram illustrating a distributed, cryptographically secure system to manage access to patient data according to an example embodiment.

FIG. 2 is a block diagram illustrating of an authorization data structure according to an example embodiment.

FIG. 3 is a block diagram illustrating of a digital request according to an example embodiment.

FIG. 4 is a flowchart illustrating a computer implemented method 400 of utilizing the ledger to reject or grant access to protected data according to an example embodiment.

FIG. 5 is a block diagram illustrating of a revocation data structure according to an example embodiment.

FIG. 6 is a flowchart illustrating a computer implemented method 600 of generating the revocation data structure to revoke one or more prior authorizations to access the protected data according to an example embodiment.

FIG. 7 is a flowchart illustrating a method of generating authorizations according to an example embodiment.

FIG. 8 is a flowchart illustrating a computer implemented method of revoking an authorization according to an example embodiment.

FIG. 9 is a flowchart illustrating a computer implemented method of fulfilling a data request according to an example embodiment.

FIG. 10 is a block schematic diagram of a computer system to implement one or more example embodiments.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

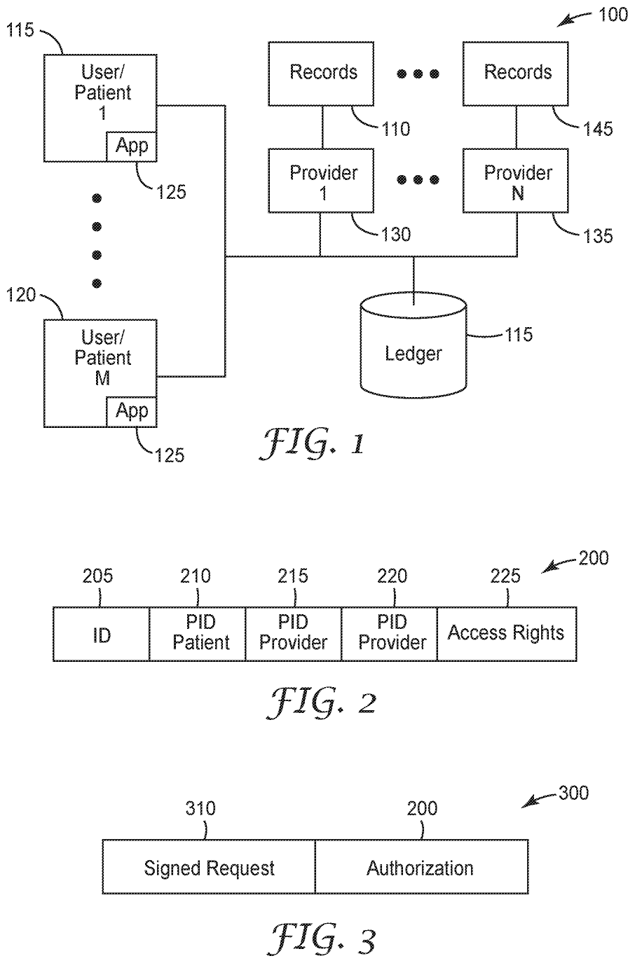

FIG. 1 is a block diagram illustrating a distributed, cryptographically secure system 100 to manage access to patient data. System 100 may be an open distributed system in one embodiment. System 100 utilizes cryptographically linked blocks in the form of a ledger 110 for data access control. Blockchain technology may be used for the ledger 110. System 100 does not expose any personal health information as it tracks revocation of prior access authorizations.

Multiple users, such as M users are represented at 115 and 120. The term “users” may be used to represent users, such as patients or representatives of patients as well as their associated computing devices that run an app 125 for use in generating authorizations to access data and revocations of such authorizations and to perform other functions as described herein. The term app includes any software or hardware combination to perform the functions.

To authorize one provider to access a patient's medical records, a patient may use the app 125 to digitally issue a cryptographically signed authorization document to those who need access to their data. The app may also be used to generate a public/private key pair to facilitate signing of the authorization. The public key may also be used to identify the patient and may be referred to as a participant identifier (PID). Providers, indicated at 130, 135, such as N providers, may also be given participant identifiers (PIDs) which may comprise a public key of a public/private key pair assigned to the provider. The provider may be a healthcare facility, a doctor, nurse, institution, or other entity to which authorization to access protected data represented as medical records 140 and 145 may be given.

The authorization generated via the app by the patient, or a representative of the patient, is assigned a universally unique identifier (UUID) to help identify each authorization without revealing patient information. Providers/institutions who have received digital authorization documents from a patient can then transmit the authorizations in a request to other providers/institutions (such as a patient's previous doctor) along with requests for the patient's medical record, which may be stored in digital form.

The request for the patient's medical record is cryptographically signed. The request can be verified or rejected by an automated computer system based on the signature, removing the need for a person to manually review the request before sending the medical or other protected data.

The system 100 is secured by having all parties publish revocation records to the shared set of cryptographically linked blocks (ledger 115), such as a public blockchain. All authorization records that need to be validated by any party at any time only need to be checked against the blockchain to see if they have been revoked; if not, then the authorization is still valid (i.e., access remains granted).

### Definitions

Revocation Ledger: an append-only ledger system where all participants have read/write access; may be, but is not necessarily, implemented as a blockchain.

Participant Identifier (PID): an identifier which designates a specific participant in the system and consists of that participant's public key; this identifier would be considered PHI when applied to a patient.

Universally Unique Identifier (UUID): an ID number, generated according to the generally accepted specifications, such that it is highly likely to be universally unique.

In one embodiment, each individual/entity such as a patient, provider, or institution has its own public/private key pair. The public key is used for identification/verification and the private key is used for signing. The public/private key pair can be generated by a variety of methods, such as app on a mobile phone.

The public/private key pair is held (stored) by each individual such as in the mobile phone app. Ideally, each individual/entity would only have a single public/private key pair in their lifetime.

When a patient wants to grant a new provider/institution access to their previous medical records, an “authorization document” is issued and signed using the patient's private key. This authorization document is the machine-readable equivalent of “I, John Doe, grant <full, partial, etc.> access to my medical record to XYZ clinic”. The authorization document is a data structure that contains, at a minimum, the following as illustrated in block form in FIG. 2 at 200:

Authorization ID 205 (a random 256-bit number which uniquely identifies the authorization being issued). The authorization ID is also referred to as the UUID;

The patient's public key or PID 210, which serves to identify the patient;

The public key or PID 215 of the provider/institution to which access is being granted;

If known, the public key or PID 220 of the previous provider/institution currently storing the patient's previous data (so the new provider/institution knows where to request data from); and

Information specifying exactly what access rights 225 are being authorized (read-only access, read/write access, full or partial record, etc.).

In operation, a patient may receive multiple provider PIDs and use the app to generate the authorization document 200 containing the UUID 205, the PID for participant granting access (patient or their healthcare proxy), a PID for participant receiving access (e.g. specialist physician, organization), a PID for the participant (e.g. primary care physician, organization, laboratory, etc.) who will release the data to the participant who receives access, specification for the type of documents and access rights being granted (read, read/write, restricted to certain types of data, etc.), and additional data may be provided, but are not required, such as effective dates (start/end) rights are being issued for, specified in Coordinated Universal Time (UTC)

The patient then signs the authorization document using their private key. Signing with the patient's private key produces a data structure, called a “digital signature.” The digital signature can be verified with patient's public key to prove that the holder of the private key “signed” the document. This digital structure is specific to the document which was signed. Any changes made to the document after the digital signature has been generated will invalidate the digital signature. The patient transmits the signed authorization document to the provider (e.g. specialist physician) who is granted access.

Next, the new provider/institution requests the patient's medical records, generating a digital request as illustrated in FIG. 3 at 300, signed with the private key of the new provider/institution. This request, bundled with the authorization document 200 signed as indicated at 310 by the patient's private key, is transmitted to the previous provider/institution storing the patient's medical data.

The previous provider/institution receiving the record request from the new provider/institution verifies the authorization document against the patient's public key, which is already stored at the previous provider/institution together with the patient's medical record, to correctly identify the right patient and medical record.

The previous provider/institution also checks the ledger 115 storing revocation records—if this authorization document UUID is not found there, it means it has not been revoked and thus is valid.

To revoke a previously issued authorization document, the patient will publish a revocation to the shared ledger. The revocation is simply the previously issued authorization ID (UUID) signed with the patient's private key.

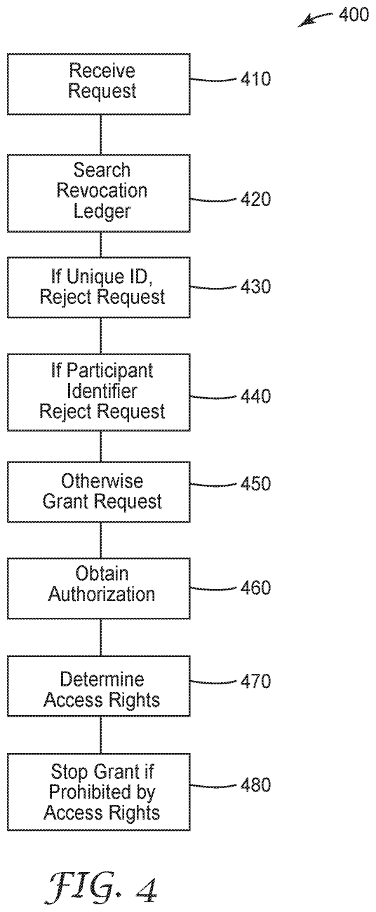

FIG. 4 is a flowchart illustrating a computer implemented method 400 of utilizing the ledger 115 to reject or grant access to protected data. Method 400 utilizes computer executed code to perform operations. At operation 410, a request is received at a provider for protected data. The request includes a unique identifier corresponding to an authorization to access the protected data and a participant identifier. One or both may be included in the request and have different effects as described below.

At operation 420, the revocation ledger 115 is searched. In one embodiment, the revocation ledger 115 comprises a set of cryptographically linked blocks containing transactions corresponding to revocations of authorizations by multiple persons to access protected data of such persons. Revocations are signed with private keys of public/private key pairs, each such pair associated with a corresponding entity including, for example, a patient or a third party trusted by the patient. The revocation ledger may comprise a blockchain ledger. The request is rejected or denied at operation 430 in response to the unique identifier being stored in the revocation ledger 115. In one embodiment, the revocation ledger 115 comprises cryptographically linked blocks of revocations of previous documents granting access to the protected data.

At operation 440, the request for protected data is denied or rejected in response to the participant identifier being stored in the revocation ledger 115. The request for the protected data is granted at operation 450 in response to neither the unique identifier nor that participant identifier being stored in the revocation ledger 115.

The participant identifier in one embodiment corresponds to the person associated with the protected data and comprises a public key associated with the person. The participant identifier may alternatively correspond to a provider that received the request for protected data. Such a participant identifier being found in the revocation ledger results in the authorization to that provider being revoked.

In one embodiment, the person is a patient, the protected data comprises health care records of the patient, and the provider comprises a health care facility that generated the request.

Method 400 may include further operations that occur either prior to checking the ledger, or after checking the ledger. At operation 460 a protected data authorization associated with the person is received. Access rights specified in the protected data authorization are determined at 470. The request is denied at operation 480 in response to the access rights prohibiting access. In one embodiment, the access rights define effective dates permitting access to the protected data authorization. The request is denied at operation 480 in response to a current date being outside the effective dates.

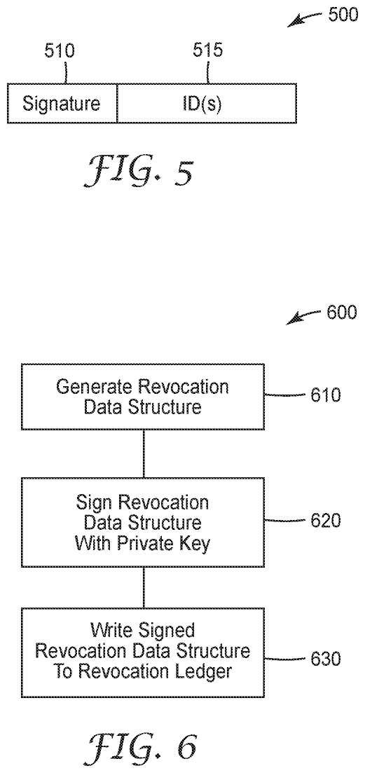

FIG. 5 is a block diagram illustrating a revocation data structure 500. The revocation data structure 500 is signed, as indicated at 510, with a private key of the person generating the revocation data structure 500. One or more IDs 515 may be included. The IDs 515 may include the UUID of the authorization document, or the PID of the person/user/patient, the PID of a provider, or any combination thereof in some embodiments. As noted in method 400, the consequences of the different PIDs vary, with the UUID simply revoking the authorization with that UUID. If the ID is that of the patient/user, all authorizations by that user are effectively revoked. If the ID that that of a provider, all authorizations permitting that provider to obtain records are revoked.

FIG. 6 is a flowchart illustrating a computer implemented method 600 of generating the revocation data structure 600 to revoke one or more prior authorizations to access the protected data. Method 600 may be performed via app 125 in some embodiments, executing on a phone, tablet, laptop, or other computing device. At operation 610, the revocation data structure is generated and contains a unique identifier corresponding to a prior authorization to access protected data of a person or a participant identifier. Both identifiers may be included in further embodiments.

The participant identifier in one embodiment corresponds to the person associated with the protected data and comprises the public key associated with the person. The participant identifier may alternatively correspond to a provider. In a further embodiment, the participant identifier corresponds to revoking all prior authorizations to access the protected data. The unique identifier corresponds to revoking the corresponding prior authorization to access the protected data.

The revocation data structure is signed at operation 620 with a private key of a public/private key pair associated with the person. At operation 630, the signed revocation data structure is written to the revocation ledger.

In one embodiment, the public/private key pair used for signing the revocation data structure and generated as described above is associated with the person. The private key is stored on the person's device that executes the app, and the public key may be shared to allow other devices to verify that the person actually signed the generated revocation.

One or more advantages of the use of the ledger for revocations may include features such as no sensitive patient data is ever shared publicly. No centralized computer system need be used, resulting in no single point of failure. The system is less vulnerable to hacking because there is no single point of failure. Patients can issue and revoke authorization records, while others can validate their authenticity, without having to trust any particular party or institution. Since revocations are expected to be an infrequent occurrence, the total cost of running the shared revocation system on a blockchain—even at global scale—is exceptionally low.

Digital identity creation may occur once, when a new participant joins the system. The creation of a new identity can occur offline and requires no communication, digital or otherwise, with an external party.

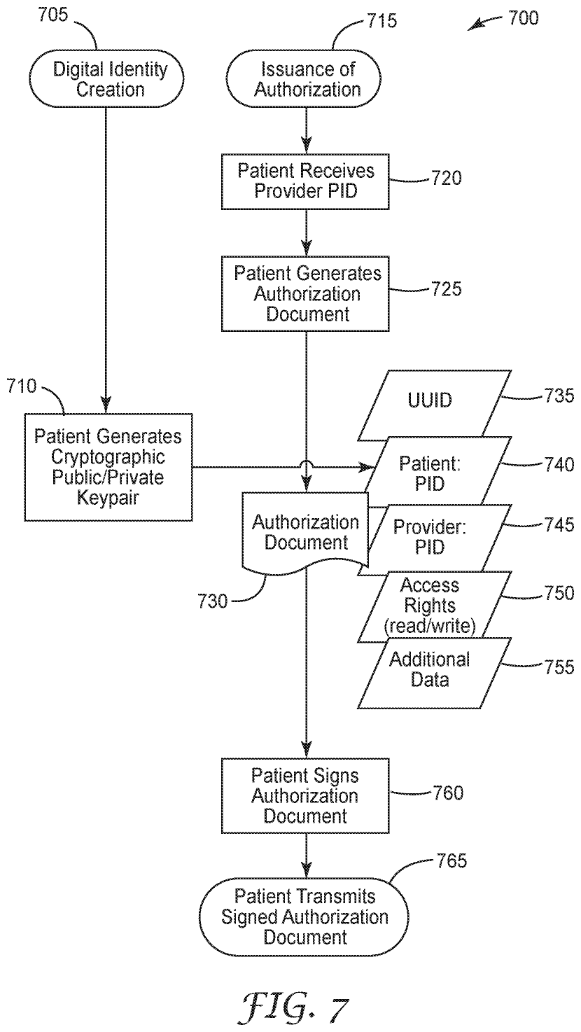

FIG. 7 is a flowchart illustrating a method 700 of generating authorizations. At operation 705, a patient may use the app 125 to generate the cryptographic public/private key pair and store the key pair securely as indicated at operation 710. The public key functions as the patient's Participant Identifier (PID) in one embodiment. Similarly, providers will also have their own digital identities, and their public keys may also serve as their Participant Identifier (PID). The PIDs are comparable to a person's driving license number, passport number, social security number, a provider's National Provider Identification number, etc., and are globally unique.

An authorization may be issued at operation 715 whenever one participant issues authorization for another participant (a new provider, such as a specialist physician) to access their data from a previous provider (e.g. primary care physician). In one example, a patient will issue an authorization to a provider. It is also possible that a patient may issue an authorization to another participant which is not a provider, such as a data broker, legal representative, family member, research institution, etc.

At operation 720, the patient may receive a PID of the provider that wishes to receive the patient's medical records from another provider. The patient then generates at operation 725 an authorization document illustrated at 730. The authorization document as described above and illustrated in FIG. 7 may include the UUID 735 of the authorization document, the patient PID 740, the receiving provider PID 745, access rights 750, and additional data 755 if desired. The patient then signs the authorization document at operation 760 and transmits the signed authorization document at 765.

In many cases, when the patient generates at operation 725 the authorization document at 730, additional revocation keys can be included in the authorization document to allow a trusted third party to revoke an authorization on behalf of the patient. The additional revocation keys can specify additional identities (e.g., for a trusted third party) which can revoke the authorization but do not gain any data access rights.

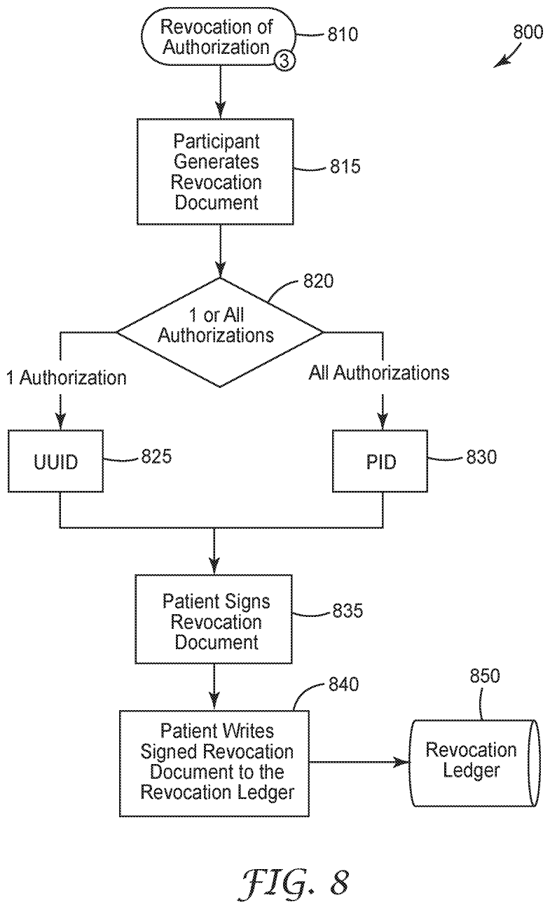

FIG. 8 is a flowchart illustrating a computer implemented method 800 of revoking an authorization beginning at operation 810. Revocation of an authorization can occur whenever a participant wants to revoke either a specific Authorization Document previously issued, or revoke all Authorization Documents associated with their PID (such as in the event that their credentials have been compromised).

Participant generates a revocation document at operation 815 containing either the UUID of the Authorization Document to revoke, or the PID of the participant (to revoke all Authorization Documents associated with the PID)—the latter may be done to revoke all the authorization given to a specific provider in case there is no longer a patient-provider relationship or all the authorizations previously granted by the patient if their credentials were compromised. At operation 820 one or all authorizations (PID or UUID) may be selected for inclusion as indicated at operations 825 UUID and 830 PID. At operation 835, the patient signs the revocation. The signed revocation document is then written at operation 840 to the revocation ledger is as indicated at 850.

In many cases, when the corresponding authorization document includes additional revocation key(s) to allow a trusted third party to revoke an authorization, instead of directly signing revocations using the patient's keys, the trusted third party can sign the revocation on behalf of the patient at operation 835. The signed revocation document is then written at operation 840 to the revocation ledger as indicated at 850. In this manner, a trusted third party, as specified by the inclusion of the additional digital identity (e.g., revocation keys) in the authorization document, can revoke an authorization on behalf of the patient without gaining any data access rights. This proxy feature substantially lowers the implementation cost and provides stronger privacy protections in practice.

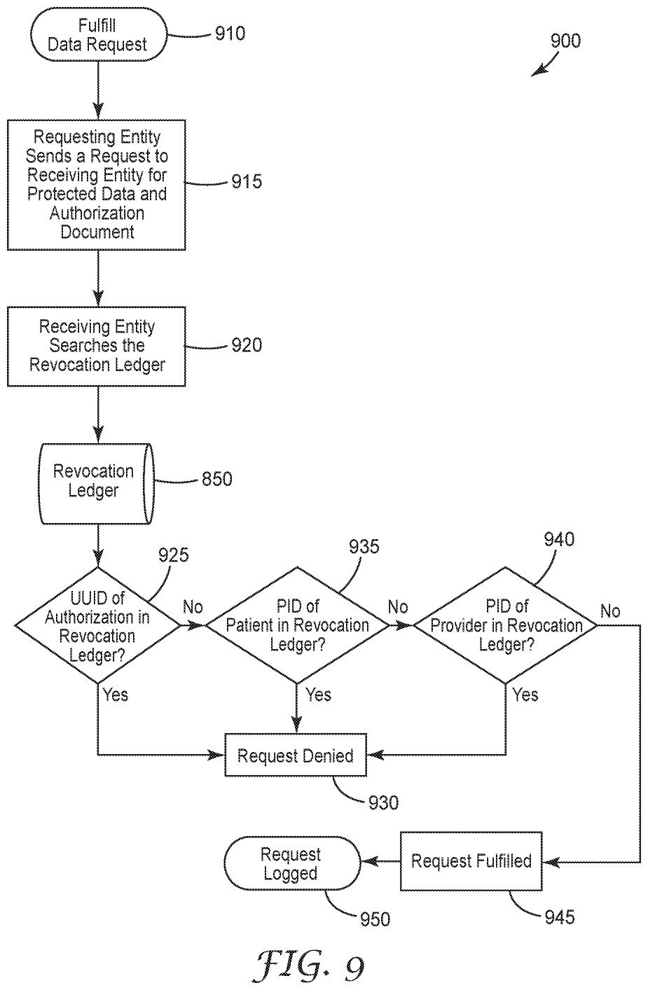

FIG. 9 is a flowchart illustrating a computer implemented method 900 of fulfilling a data request starting at operation 9190. Verification of a previously issued authorization may occur each time a request is issued to release medical records from one entity to another. A requesting entity at operation 915 sends a request to a receiving entity for protected data, such as a portion of a patient's medical record.

The receiving entity searches or initiates a search at operation 920 of the revocation ledger 850 for a revocation document matching any of the following as indicated at respective operations identified:

UUID of the Authorization Document at operation 925, with the revocation being signed by the grantor, grantee, or delegator.

PID of the grantor at operation 935, signed by the grantor.

PID of the grantee at operation 940, signed by the grantee.

PID of the delegator, also at operation 940, signed by the delegator.

If any revocation document matching the above criteria is found, the authorization document is considered to be revoked and the request is not fulfilled as indicated by request denied operation 930. Otherwise, the request is fulfilled as indicated at operation 945.

If the authorization document contains effective dates which do not include the current date, then the authorization document is considered invalid and the request is not fulfilled at 950.

If the Authorization Document is valid (has not failed a previous step), then the request is fulfilled.

Whether the request is fulfilled or rejected, the request and the provided Authorization Document may be logged at operation 950. Access to logs shall be made available to participants with authorization to see them, so that they can audit who is accessing their data and how.

Registration and identity mapping may occur each time a patient visits a new provider for the first time after creating their digital identity. In the registration and identity matching process, a provider receives the patient's Participant Identifier (PID) and connects it to their medical record, verifying their identity using appropriate legal documents as necessary. Exactly how the provider conducts identity matching and stores the patient's UID is outside the scope of our patent and may vary in implementation; what is important is that the provider links all relevant medical records back to the PID, so that they can be retrieved when an authorized user requests them in the future.

The following examples are provided for illustrative purposes only and are not exhaustive of all scenarios.

In-Person Registration:

Patient appears in-person at a provider's office for the first time after creating their digital identity.

Patient provides appropriate legal documentation (such as driver's license, passport, state-issued ID card, etc.) to prove their identity and allow for matching with previous records.

Patient provides their PID to the provider (possibly by issuing an Authorization Document, but not necessarily).

Provider stores the patient's PID and links it to past medical records, making them electronically accessible.

Registration Via Patient Portal:

In some cases, the patient may have already proved their identity to a provider and registered with some sort of online “patient portal”. In these cases, the patient's identity has already been verified and connected to existing medical records. To simplify the registration, the provider could modify their patient portal to allow the patient to log in and submit their PID online, so that is linked to their medical records for future access.

Chained Authorizations:

In some use cases, it will be necessary for a participant to delegate a portion of their access rights on to another participant. For example, a provider may need to provide access to patient data to a business associate, such as a data broker. In such a case, a participant who has received an authorization document may issue a delegate authorization document to pass a portion of their access rights on to another participant. This allows the associate to access applicable data, while protecting the patient's ability to control how their data is used and distributed. This design also enables participants to track data and authorization provenance (which may be required for audit or regulatory compliance). In one embodiment, a delegate authorization document may be created in the same manner as data access authorization documents and may also be revoked utilizing the ledger 115 and associated revocations.

The delegate authorization document will have the same format as a regular authorization document, except that it will also contain a field specifying the PID of the participant who is delegating their access, as well as a copy of the original authorization document. Multiple levels of delegation are supported, forming a chain of trust linking the participant accessing data back to the patient's original issuance of permission to access the data. A revocation issued for any authorization within the chain will automatically cancel all authorizations further down the chain which depend on it.

Revocation of an authorization to access medical records has previously been done by paper forms or PDF forms and transmitted by fax or electronically as PDF in a non-machine-readable format. Transmitting revocations to all parties who have received the corresponding authorization in the past or will do so in the future is impossible using paper, fax or current electronic transmission systems. We propose a novel solution that makes the revocations implementable in a reliable and automated way. Our novel solution stores revocation record on a publicly available ledger, which ensures its availability and authenticity.

System 100 may have one or more unique advantages compared to other methods that involve the use of cryptographically linked blocks in healthcare:

Compared to other methods that propose storing patient information or pointers to patient information stored elsewhere, system 100 does not store any individually identifiable patient information on the ledger. system 100 does not store any pointers to individually identifiable patient information on the ledger. Pointers to previous authorization records are shared via the UUID. Hence, patient privacy and confidentiality are protected.

By querying the revocation records, one cannot tell the identity of the patient or the specialty of the provider who recorded them on the ledger. In solutions where provider organizations would write information about the patient to the blockchain, one can infer the patient's location, travel patterns or sensitive health conditions simply by noticing the provider who has added data to a given patient's record, their location and specialty. System 100 does not have such risks of leak of sensitive health information.

Storing patient data or pointers to health care data introduces performance issues when the data need to be queried. This can cause delays at the point of care. However, system 100 does not involve querying the ledger at the point of care. Checking if an authorization has been revoked needs to be done only by the health information department before releasing information, and hence does not affect the patient encounter at the point of care.

### Example: John Doe Gets a New Doctor

A patient, John Doe, wants to set up electronic access to his medical records, so that he can easily view his data and transfer it between doctors.

John starts by downloading the app 125 which provides an easy-to-use interface for generating the necessary Participant ID number (PID) and cryptographic keys he needs to get started. The app 125 can generate these on its own, without needing to communicate with any external servers or John can enter the keys if he already has them.

Once John has generated this data, on his next visit to this doctor, he provides his Participant ID (PID) number to his doctor. The doctor adds John's PID to his electronic medical record. Now, John's current medical records are linked to his PID, so they can be easily searched for and retrieved in the future.

A few months later, John decides to get a new doctor.

On his first visit to his new doctor, John again provides his PID number. He also provides a digital Authorization Document, which he can also generate with the app. This Authorization Document gives his new doctor's computer system permission to automatically contact his old doctor's computer system and download his medical records. Since the Authorization Document is signed with a verifiable cryptographic signature, his old doctor's computer system can automatically check it and verify that it is valid before transmitting his records to the new doctor.

If, in the future, John may decide to again get a new doctor. When this happens, he can easily create another Authorization Document allowing his new doctor to access his medical records from both of his previous doctors. Also, if he wants to, John can revoke any of those Authorization Documents at any time, cutting off a specific provider's access to his medical records. He may want to do this, for example, if one of his doctors is the victim of a cyberattack or computer system breach and he wants to protect his data.

Utilizing the described methods for data access control, John can seamlessly transfer his medical records between different medical providers, making them available to new providers or revoking access instantly.

In contrast, without such a technology, a patient likely would have had to visit their previous doctor(s) and personally get copies of their medical records and bring them to the new doctor or sign a paper-based or PDF authorization form at his new doctor's office, which gets faxed to the previous doctor's office. The previous doctor's office does not have a way of verifying if the authorization is valid (or has been revoked) while faxing the medical records to the new doctor's office. Someone would have then had to spend a great deal of time entering the information from these records into the new doctor's computer system. This prior process is slow, inefficient, and does not scale.

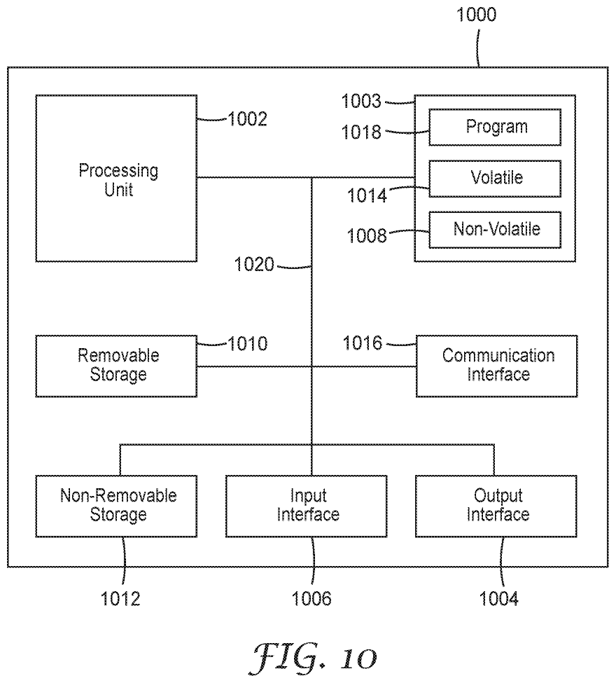

FIG. 10 is a block schematic diagram of a computer system 1000 to implement system 100 including user devices, provider devices, the ledger, and for performing methods and algorithms according to example embodiments. All components need not be used in various embodiments.

One example computing device in the form of a computer 1000 may include a processing unit 1002, memory 1003, removable storage 1010, and non-removable storage 1012. Although the example computing device is illustrated and described as computer 1000, the computing device may be in different forms in different embodiments. For example, the computing device may instead be a smartphone, a tablet, smartwatch, smart storage device (SSD), or other computing device including the same or similar elements as illustrated and described with regard to FIG. 10. Devices, such as smartphones, tablets, and smartwatches, are generally collectively referred to as mobile devices or user equipment.

Although the various data storage elements are illustrated as part of the computer 1000, the storage may also or alternatively include cloud-based storage accessible via a network, such as the Internet or server based storage. Note also that an SSD may include a processor on which the parser may be run, allowing transfer of parsed, filtered data through I/O channels between the SSD and main memory.

Memory 1003 may include volatile memory 1014 and non-volatile memory 1008. Computer 1000 may include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1000 may include or have access to a computing environment that includes input interface 1006, output interface 1004, and a communication interface 1016. Output interface 1004 may include a display device, such as a touchscreen, that also may serve as an input device. The input interface 1006 may include one or more of a touchscreen, touchpad, mouse, keyboard, camera, one or more device-specific buttons, one or more sensors integrated within or coupled via wired or wireless data connections to the computer 1000, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 1000 are connected with a system bus 1020.

Computer-readable instructions stored on a computer-readable medium are executable by the processing unit 1002 of the computer 1000, such as a program 1018. The program 1018 in some embodiments comprises software to implement the app 125 and one or more methods for generating and processing requests, accessing the ledger and other methods described herein. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 1018 along with the workspace manager 1022 may be used to cause processing unit 1002 to perform one or more methods or algorithms described herein.

### EXAMPLES

1. A computer implemented method includes receiving a request at a provider for protected data, the request including a unique identifier corresponding to an authorization to access the protected data or a participant identifier, searching a revocation ledger,

denying the request for protected data in response to the unique identifier being stored in the revocation ledger wherein the revocation ledger comprises cryptographically linked blocks of revocations of previous documents granting access to the protected data, denying the request for protected data in response to the participant identifier being stored in the revocation ledger, and granting the request for the protected data in response to neither the unique identifier nor that participant identifier being stored in the revocation ledger.

2. The method of example 1 wherein the participant identifier corresponds to the person associated with the protected data and comprises a public key associated with the person.

3. The method of any of examples 1-2 wherein the participant identifier corresponds to a provider that received the request for protected data.

4. The method of any of examples 1-3 wherein the person comprises a patient, the protected data comprises health care records of the patient, and the provider comprises a health care facility that generated the request.

5. The method of any of examples 1-4 and further including obtaining a protected data authorization associated with the person, determining access rights specified in the protected data authorization, and denying the request in response to the access rights prohibiting access.

6. The method of any of examples 1-5 and further including obtaining a protected data authorization associated with the person, determining effective dates permitting access to the protected data authorization, and denying the request in response to a current date being outside the effective dates.

7. The method of any of examples 1-6 wherein the revocation ledger comprises a set of cryptographically linked blocks containing transactions corresponding to revocations of authorizations by multiple persons to access protected data of such persons.

8. The method of example 7 wherein such revocations are signed with private keys of public/private key pairs, each such pair associated with a corresponding entity including a trusted third party.

9. The method of any of examples 1-8 wherein the revocation ledger comprises a blockchain ledger.

10. A machine-readable storage device has instructions for execution by a processor of a machine to cause the processor to perform operations to perform a method, the operations including receiving a request at a provider for protected data, the request including a unique identifier corresponding to an authorization to access the protected data or a participant identifier, searching a revocation ledger, denying the request for protected data in response to the unique identifier being stored in the revocation ledger wherein the revocation ledger comprises cryptographically linked blocks of revocations of previous documents granting access to the protected data, denying the request for protected data in response to the participant identifier being stored in the revocation ledger, and granting the request for the protected data in response to neither the unique identifier nor that participant identifier being stored in the revocation ledger.

11. The device of example 10 wherein the participant identifier corresponds to the person associated with the protected data and comprises a public key associated with the person.

12. The device of any of examples 10-11 wherein the person comprises a patient, the protected data comprises health care records of the patient, and the provider comprises a health care facility that generated the request.

13. The device of any of examples 10-12 and further including obtaining a protected data authorization associated with the person, determining access rights specified in the protected data authorization, and denying the request in response to the access rights prohibiting access.

14. The device of any of examples 10-13 and further including obtaining a protected data authorization associated with the person, determining effective dates permitting access to the protected data authorization, and denying the request in response to a current date being outside the effective dates.

15. The device of any of examples 10-14 wherein the revocation ledger comprises a set of cryptographically linked blocks containing transactions corresponding to revocations of authorizations by multiple persons to access protected data of such persons.

16. A device includes a processor and a memory device coupled to the processor and having a program stored thereon for execution by the processor to perform operations. The operations include receiving a request at a provider for protected data, the request including a unique identifier corresponding to an authorization to access the protected data or a participant identifier, searching a revocation ledger, denying the request for protected data in response to the unique identifier being stored in the revocation ledger wherein the revocation ledger comprises cryptographically linked blocks of revocations of previous documents granting access to the protected data, denying the request for protected data in response to the participant identifier being stored in the revocation ledger, and granting the request for the protected data in response to neither the unique identifier nor that participant identifier being stored in the revocation ledger.

17. The device of example 16 wherein the participant identifier corresponds to the person associated with the protected data and comprises a public key associated with the person.

18. The device of any of examples 16-17 wherein the person is a patient, the protected data are health care records of the patient, and the provider is a health care facility that generated the request.

19. The device of any of examples 16-18 wherein the operations further include obtaining a protected data authorization associated with the person, determining access rights specified in the protected data authorization, and denying the request in response to the access rights prohibiting access.

20. The device of any of examples 16-19 wherein the operations further include obtaining a protected data authorization associated with the person determining effective dates permitting access to the protected data authorization, and denying the request in response to a current date being outside the effective dates.

21. A computer implemented method comprising: generating a revocation data structure containing a unique identifier corresponding to a prior authorization to access protected data of a person or a participant identifier; signing the revocation data structure with a private key of a public/private key pair associated with the person; and writing the signed revocation data structure to a revocation ledger.

22. The method of example 21 and further comprising generating the public/private key pair prior to signing the revocation data structure.

23. The method of any of examples 21-22 wherein the participant identifier corresponds to the person associated with the protected data and comprises the public key associated with the person.

24. The method of example 23 wherein the participant identifier corresponds to revoking all prior authorizations to access the protected data.

25. The method of any of examples 21-24 wherein the unique identifier corresponds to revoking the corresponding prior authorization to access the protected data.

26. The method of any of examples 21-25 wherein the participant identifier corresponds to a provider.

27. The method of example 26 wherein the person comprises a patient, the protected data comprises health care records of the patient, and the provider comprises a health care facility to which authorization to access the protected data was previously provided.

28. The method of any of examples 21-27 wherein the revocation ledger comprises a set of cryptographically linked blocks containing transactions corresponding to revocations of authorizations by multiple persons to access protected data of such persons.

29. The method of any of examples 21-28 wherein the revocation ledger comprises a blockchain ledger.

30. The method of any of examples 21-29 and further comprising generating an authorization to access protected data, the authorization including and authorization unique identifier, a patient ID, a granting access provider ID, and a receiving access provider ID.

31. The method of example 30 wherein the authorization further comprises a specification of access rights.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A computer implemented method comprising:
receiving a request at a provider for protected data, the request including a unique identifier corresponding to an authorization to access the protected data or a participant identifier;
searching a revocation ledger;
denying the request for protected data in response to the unique identifier being stored in the revocation ledger wherein the revocation ledger comprises cryptographically linked blocks of revocations of previous documents granting access to the protected data;
denying the request for protected data in response to the participant identifier being stored in the revocation ledger; and
granting the request for the protected data in response to neither the unique identifier nor that participant identifier being stored in the revocation ledger.

2. The method of claim 1 wherein the participant identifier corresponds to the person associated with the protected data and comprises a public key associated with the person.

3. The method of claim 1 wherein the participant identifier corresponds to a provider that received the request for protected data.

4. The method of claim 1 wherein the person comprises a patient, the protected data comprises health care records of the patient, and the provider comprises a health care facility that generated the request.

5. The method of claim 1 and further comprising:
obtaining a protected data authorization associated with the person;
determining access rights specified in the protected data authorization; and
denying the request in response to the access rights prohibiting access.

6. The method of claim 1 and further comprising:
obtaining a protected data authorization associated with the person;
determining effective dates permitting access to the protected data authorization; and
denying the request in response to a current date being outside the effective dates.

7. The method of claim 1 wherein the revocation ledger comprises a set of cryptographically linked blocks containing transactions corresponding to revocations of authorizations by multiple persons to access protected data of such persons.

8. The method of claim 7 wherein such revocations are signed with private keys of public/private key pairs, each such pair associated with a corresponding entity including a trusted third party.

9. The method of claim 1 wherein the revocation ledger comprises a blockchain ledger.

10. A machine-readable storage device having instructions for execution by a processor of a machine to cause the processor to perform operations to perform a method, the operations comprising:
receiving a request at a provider for protected data, the request including a unique identifier corresponding to an authorization to access the protected data or a participant identifier;
searching a revocation ledger;
denying the request for protected data in response to the unique identifier being stored in the revocation ledger wherein the revocation ledger comprises cryptographically linked blocks of revocations of previous documents granting access to the protected data;
denying the request for protected data in response to the participant identifier being stored in the revocation ledger; and
granting the request for the protected data in response to neither the unique identifier nor that participant identifier being stored in the revocation ledger.

11. The device of claim 10 wherein the participant identifier corresponds to the person associated with the protected data and comprises a public key associated with the person.

12. The device of claim 10 wherein the person comprises a patient, the protected data comprises health care records of the patient, and the provider comprises a health care facility that generated the request.

13. The device of claim 10 and further comprising:
obtaining a protected data authorization associated with the person;
determining access rights specified in the protected data authorization; and
denying the request in response to the access rights prohibiting access.

14. The device of claim 10 and further comprising:
obtaining a protected data authorization associated with the person;
determining effective dates permitting access to the protected data authorization; and
denying the request in response to a current date being outside the effective dates.

15. The device of claim 10 wherein the revocation ledger comprises a set of cryptographically linked blocks containing transactions corresponding to revocations of authorizations by multiple persons to access protected data of such persons.

16. A device comprising:
a processor; and
a memory device coupled to the processor and having a program stored thereon for execution by the processor to perform operations comprising:
receiving a request at a provider for protected data, the request including a unique identifier corresponding to an authorization to access the protected data or a participant identifier;
searching a revocation ledger;
denying the request for protected data in response to the unique identifier being stored in the revocation ledger wherein the revocation ledger comprises cryptographically linked blocks of revocations of previous documents granting access to the protected data;
denying the request for protected data in response to the participant identifier being stored in the revocation ledger; and
granting the request for the protected data in response to neither the unique identifier nor that participant identifier being stored in the revocation ledger.

17. The device of claim 16 wherein the participant identifier corresponds to the person associated with the protected data and comprises a public key associated with the person.

18. The device of claim 16 wherein the person comprises a patient, the protected data comprises health care records of the patient, and the provider comprises a health care facility that generated the request.

19. The device of claim 16 wherein the operations further comprise:
obtaining a protected data authorization associated with the person;
determining access rights specified in the protected data authorization; and
denying the request in response to the access rights prohibiting access.

20. The device of claim 16 wherein the operations further comprise:
obtaining a protected data authorization associated with the person;
determining effective dates permitting access to the protected data authorization; and
denying the request in response to a current date being outside the effective dates.
